(19) United States
(12) Patent Application Publication
(10) Pub. No.: US 2004/0073129 A1
(43) Pub. Date: Apr. 15, 2004

(54) EEG system for time-scaling presentations

(75) Inventors: Samuel J. Caldwell, Shreveport, LA (US); Taka-aki Tanaka, Tokyo (JP); Yoku Ogawa, Tokyo (JP)

Correspondence Address: PATENT LAW OFFICES OF DAVID MILLERS, 6560 Ashfield Court, San Jose, CA, 95120, US

(73) Assignee: SSI Corporation

(21) Appl. No.: 10/272,514

(22) Filed: Oct. 15, 2002

(51) Int. Cl. A61B 5/04

(52) U.S. Cl. 600/544

(57) ABSTRACT

A data acquisition unit for an EEG system includes pliant electrodes and/or a wireless transmitter that permit use of the EEG system without electrolyte gels or solutions and/or connecting wires. The electrodes can use a conductive fabric or a conductive rubber material that is dry or damp and mounted in a rigid structure that plugs into a socket on a headset. A feedback unit in the EEG system, which receives and processes the data from data acquisition unit, can be a high power, high performance processing system that implements complex feedback presentations and control functions based on analysis of the EEG data. In one embodiment, the feedback system controls a presentation player and adjusts a playback rate according to the sensed brain activity or synchrony between left and right brain activity. A PWM signal can control the time scale of the presentation.

### BACKGROUND

Electroencephalograms (EEGs) are known for electrically measuring brainwave activity in medical and consumer applications. As a consumer device, an EEG generally provides feedback (e.g., a visual or audible presentation) enabling a user to observe his or her brain activity. Some people observing the EEG output can develop a degree of control over the brain activities that produce specific EEG output signals, and many people are interested in achieving such control. These skills are of particular importance to people that have lost nervous system control or motor skills. See for example, Chase, “Mind over Muscles”, Technology Review, March/April 2000.

Standalone EEG units available to the general public have been relatively simple systems with LED lights indicating a dominant wave pattern (e.g., distinguishing between alpha-rhythm and beta-rhythm brainwaves) and numeric displays indicating data such as brainwave frequency and amplitude. Beeps or activation of LEDs provide feedback indicating various brainwave states, e.g., a wave pattern reaching a selected threshold, falling below a selected threshold, or meeting some other criteria. More advanced standalone consumer EEG systems may have components such as LCD displays and audio systems to enhance visual and audio feedback, but the processing power needed to acquire and plot EEG data in real time has limited the appearance and function of feedback from standalone EEGs. For example, EEG visual presentations have generally been on black-and-white screens with coarse granularity. Also, such systems infrequently update data, e.g., approximately once every second.

In recent years, some consumer EEG systems have added computer interfaces that permit a personal computer (PC) to analyze and display EEG data. Such systems generally have more processing power than standalone EEG systems and can display EEG data in a manner previously unavailable to standalone EEG systems, but EEG systems requiring a PC must address compatibility issues for the variety of PC systems and configurations. Additionally, the mobility of the PC limits a user's mobility, and connections between the PC and the user must be controlled to minimize the risk of electrical shock.

EEG data acquisition generally requires attaching electrodes to the user's head. These electrodes can be made of various metals and connected to wires that plug into the EEG analysis or display system. The wires, which are traditionally worn dangling from the user's head, tend to restrict the user's movement and can easily snag when the user moves. To hold the electrodes in place and reduce the chances of snagging wires, electrodes can be planted on the underside of a cap similar to a swimming cap, with a number of electrodes placed in a suitable pattern for data acquisition. However, whether the electrodes are used individually or in a cap, the user remains tethered to the EEG system through the wires running from each electrode.

Another inconvenience for EEG systems is the electrolyte cream or other solution that is generally required between the electrodes and the user's forehead or scalp to obtain good signal quality. The electrolyte cream from the electrodes generally sticks to the user's head and hair after use of the EEG, requiring the user to wash or shower after using the EEG system. Some EEG electrodes can be used with saline solutions that may be less messy than electrolyte creams but are still an inconvenience when using EEG systems. Further, use of a saline solution in electrodes has required preparing the solution with the correct salt concentration and soaking the electrodes for a period of time. The electrodes are then used wet and dripping solution.

A safety concern for EEG systems is the risk of electric shock. The electrodes provide good electrical connection of the user to the EEG system, and the EEG system generally requires a relatively robust power source to run a processor, video display, and audio system. Traditionally, consumer EEG systems have required careful management of the power components to avoid malfunctions that could shock or electrocute the user.

In view of the limitations of current systems, a consumer EEG system is sought that avoids the inconvenience of electrolyte creams and the movement restrictions of current EEG systems, provides high EEG data quality, and is capable of advanced processing and multimedia presentation of the EEG data.

In what has been a separate area of technology, time scaling (e.g., time compression or expansion) of a digital audio signal changes the play rate of a recorded audio signal without altering the perceived pitch of the audio. Accordingly, a listener using a presentation system having time scaling capabilities can speed up the audio to more quickly receive information or slow down the audio to more slowly receive information, while the time scaling preserves the pitch of the original audio to make the information easier to listen to and understand. Ideally, a presentation system with time scaling capabilities should give the listener control of the play rate or time scale of a presentation so that the listener can select a rate that corresponds to the complexity of the information being presented and the amount of attention that the listener is devoting to the presentation.

People using time-scaling presentations systems can train themselves to understand information from presentations played at higher rates. Accordingly, after some use of time scaling systems, users can often play and understand presentations at higher rates than they could when they first began using the presentation system. Development of this skill may speed up the thought processes involved in recognizing and understanding the presentations and may additionally have beneficial effects in improving the speed of other thought processes. Accordingly, many people are interested in using presentations systems with time scaling capability not only for the advantage of being able to receive information efficiently at their selected rate but also for the chance of improving mental processes.

### SUMMARY

In accordance with an aspect of the invention, a consumer EEG system employs flexible dry or semidry electrodes that sense electrical signals without requiring an electrolyte cream or solution. In one embodiment, the flexible electrodes use a cloth impregnated with conductive components, e.g., a metal or a metal compound such as silver or silver chloride. The conductive cloth can be used totally dry or dampened with tap water. In an alternative embodiment of the invention, the flexible electrodes use conductive rubber or conductive elastomer. The flexible electrodes conform to a user's head and provide sufficient sensitivity for EEG measurements without the mess involved with electrodes requiring electrolyte creams or solutions.

In accordance with another aspect of the invention, a headset includes fixtures that position the electrodes securely against a user for good electrical sensing operations. One embodiment of a fixture includes a cup containing a foam rubber or other compressible material to which a flexible electrode is attached. A wire connected to the flexible electrode extends through the compressible material to a contact or a pin on the cup. The contact or pin and the cup plugs into a socket on the headset to provide an electrical connection between the electrode and data acquisition and transmission circuitry mounted on the headset. The electrode is thus easily removable from the headset for cleaning or replacement.

In accordance with another aspect of the invention, an EEG system uses a data acquisition unit or headset having a multi-channel wireless connection to a feedback system. The data acquisition unit generally can be a low power (e.g., battery operated) system that performs data acquisition and transmission functions such as signal amplification, filtering, analog-to-digital conversion, and formatting of multi-channel signals to preserve the quality of EEG data before transmission. The multiple channels allow for analysis of left and right EEG measurements for evaluation of the synchrony between the activity on the left and right sides of the user's brain. The wireless communication, which can be infrared or radio frequency, for example, leaves the user free to move about (within the transmission range of the EEG system).

One configuration of the data acquisition unit is a headset including three electrodes contacting a subject's forehead and an electrode clipped to a user's ear. Voltages on the left and right forehead electrodes relative respectively provide left and right input signals, and the ear electrode provides a shared active signal. An amplifier module includes two balanced differential amplifiers. One balance differential amplifier amplifies a voltage difference between the left input signal and a shared active signal, and the other balanced differential amplifier amplifies a voltage difference between the right input signal and a shared active signal. The amplified signals are converted to left and right digital data streams that the data acquisition unit transmits to a feedback system. Software in the feedback system can process and use the brain activity signals in a variety of ways including but not limited to displaying waveforms or other visual representations of brain activity or changing the operating parameters of an external device according to the brain activity signals.

The wireless connectivity of a multi-channel data acquisition unit eliminates direct electrical connections of the feedback unit to the user, permitting the feedback unit to be upgraded into a high-powered, high-performance computing device without increasing the electrical shock hazard. In particular, the feedback unit can use household electricity or enough power for a powerful processor running an operating system, a full-color screen, stereo amplifiers and speakers, and data storage devices. These capabilities are further upgradeable at the same pace as the advance in the performance of computers. The feedback system can accordingly provide rich multimedia content greatly superior to prior standalone EEG systems, which provide only rudimentary sounds and numerical displays. Further, the standalone feedback unit in accordance with the invention, unlike consumer EEG systems requiring interfaces to personal computers, does not need to accommodate the variety of software, operating system, and hardware configuration in personal computers.

The feedback system can also store and run software programs including the user-selected display or feedback routines and system control operations. For example, a user may select to watch real-time filtered EEG data flowing across the screen. Alternatively, the user may attempt to use brainwaves to control the playback (e.g., speed, play/pause, or sequencing) of 3D animation and audio or to control software or any software-controlled electrical or mechanical system.

In accordance with yet another aspect of the invention, the feedback system analyzes the brain activity signals and generates a control signal for control of an external device. In one specific embodiment, the external device is a presentation system having time-scaling capabilities, and the control signal from the feedback system adjusts the time scale, volume, or other operating parameters of a presentation. With such features, the user can observe how playing a presentation at different time scales affects brain activity as displayed on the feedback system and/or attempt to learn to control an external device via the brain activity.

Even if a user does not have voluntary control of the brain activity signal, the presentation system can still interpret the brain activity signal and change the time scale if the brain activity signal indicates that the time scale is too fast or too slow. In particular, the feedback system can measure filtered EEG amplitudes associated with heightened states of awareness or synchrony between brain activity measured for the left and right sides of the user's brain to determine whether the presentation speed is optimal for learning. If the filtered EEG amplitudes or the synchrony measurements indicate the current presentation playback speed is not optimal, the feedback system can automatically adjust the playback speed according to the user's needs.

In one embodiment of the invention, a pulse width modulated (PWM) control signal between the feedback system and an external device being controlled has a pulse width indicating a control value for the external device. In particular, when the external device is a presentation system with time scaling capabilities, the pulse width of the control signal selects the time scale of the presentation system. By changing the pulse width, the feedback system can cause time scale to immediately jump any desired time scale. More generally, the pulse width can represent an operating parameter of the external system being controlled. The PWM control signal provides simple data communication without the need for complex synchronization or data transmission protocols.

### BRIEF DESCRIPTION OF THE DRAWINGS

FIG. 1 is a block diagram of a presentation system in accordance with an embodiment of the invention.

FIGS. 2A, 2B, and 2C are side, transparent top, and internal views of a headset in accordance with an embodiment of the invention.

FIGS. 3A and 3B are respectively a cross-sectional view and a back view of a fixture for an EEG electrode in accordance with an embodiment of the invention.

FIG. 4 is a block diagram of a consumer EEG system using an interface to a personal computer.

FIG. 5 is a block diagram of a consumer EEG system using a standalone feedback system capable of controlling a presentation player.

FIG. 6 is a flow diagram of a control process for an EEG system including a presentation system with audio time scaling capabilities.

Use of the same reference symbols in different figures indicates similar or identical items.

### DETALED DESCRIPTION

In accordance with an aspect of the invention, a wireless EEG data acquisition unit has dry or semidry flexible electrodes and provides a multi-channel digital data stream to a feedback system. The data acquisition unit can be mounted in a headset having socketed electrodes that allow easy removal and cleaning of the electrodes and automatic positioning of the electrodes. The feedback system can process and display EEG data or analyze the EEG data to generate a control signal. In one embodiment, the feedback system includes or connects to a presentation system with time-scaling capabilities. The feedback system can provide multimedia feedback to a user to represent the sensed brain activity and/or analyze the sensed activity to determine how to control the presentation system. In one aspect of the invention, an operating parameter such as the time scale of playback from the presentation system is set according to synchrony of measured brain activity on the left and right sides of the user's brain. A pulse width modulated (PWM) control signal from the feedback system provides a simple method for control of the time scale or other operating parameters of the presentation system. With such features, the user can observe the effect of different time scales on brain function and may acquire voluntary control of the EEG signals to control external systems.

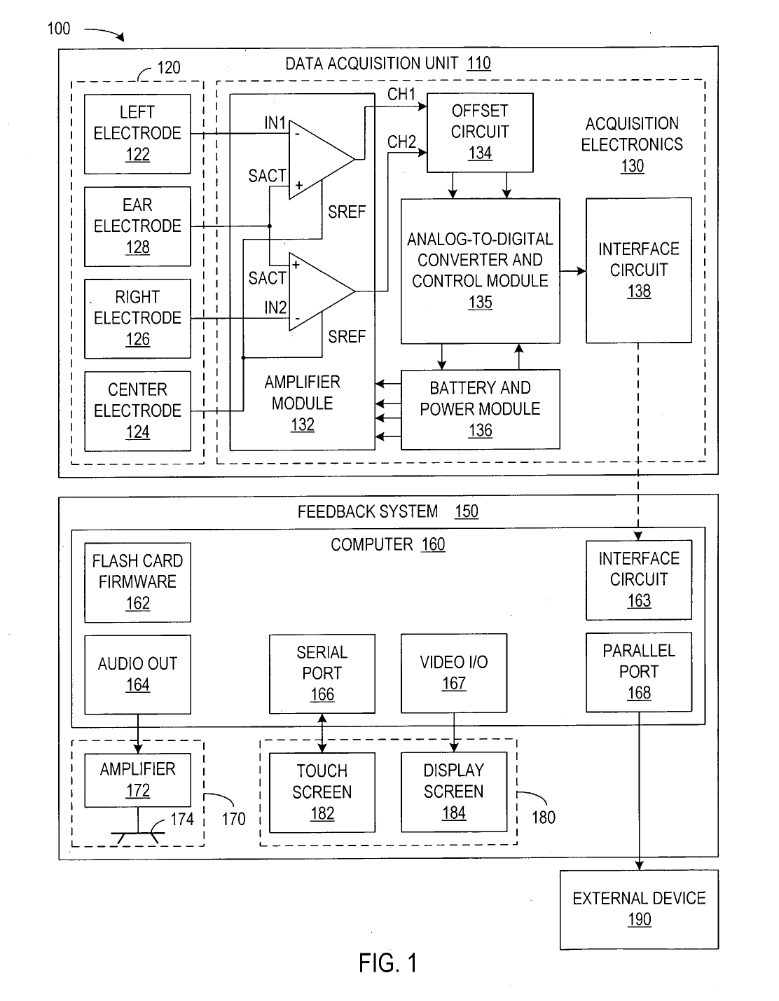

FIG. 1 is a block diagram of an EEG system 100 including a data acquisition unit 110 and a feedback unit 150 in accordance with an embodiment of the invention. Data acquisition unit 110 includes headgear (not shown) that a user wears. In an exemplary embodiment of the invention, the headgear includes attached sensing electrodes 120 and acquisition electronics 130.

Sensing electrodes 120 in FIG. 1 include four electrodes 122, 124, 126, and 128. Electrodes 122, 124, and 126 are pliant electrodes and respectively contact left, center, and right portions of a user's forehead. In one embodiment of the invention, each of electrodes 122, 124, and 126 has a cloth or fabric covering that is impregnated with conductive particles (e.g., silver or silver chloride particles) to provide a low resistance contact against the user's forehead. In another embodiment, pliant electrodes 122, 124, and 126 use a conductive rubber such as one of the electrically conductive elastomers available from Laid Technologies of Delaware Water Gap, Pa. The conductive elastomers preferably have high conductivity and low offset voltage characteristics, making them suitable for EEG electrodes. The offset voltage of electrode materials result from chemical or electrolytic interactions between skin and electrodes that create an offset voltage that makes measurement of EEG voltages difficult, but the fabric and rubber electrodes in accordance with the present invention have a low offset voltage that permits their use without electrolyte cream or solution.

Pliant electrodes 122, 124, and 126 can be used dry or dampened with tap water, unlike prior EEG systems that have required conductive paste or gel or saline solution between the electrodes and the user. When dry, pliant electrodes 122, 124, and 126 have a high conductivity that permits sensing the small amplitude signals associated with brain waves. However, dampening pliant electrodes with ordinary tap water can further improve conductivity, without the inconvenience or mess of creams, gels, or solutions. Pliant electrodes 122, 124, and 126, whether having a cloth or rubber surface, are pasteless in that EEG potentials are sensed without the mess and inconvenience of a conductive electrolyte gels, pastes, creams, or solutions such as is normally required for EEG measurements.

Left electrode 122 and right electrode 126 provide left and right signals IN1 and IN2 for a dual channel measurement of brainwave activity. Central electrode 124 provides a shared reference SREF, which serves as a reference signal for both balanced differential amplifiers in acquisition electronics 130.

Electrode 128 clips to one of the user's ears or otherwise contacts a portion of the user's body not significantly subject to electrical variations caused by brainwaves or muscle activity. Electrode 128 thus provides a shared active signal SACT for measurement of the left and right brainwave signals.

Acquisition electronics 130 includes an amplifier module 132, an offset circuit 134, a control module 135, a power module 136, and an interface circuit 138.

Amplifier module 132 contains two balanced differential amplifiers, which can be of any of the types known in the art for amplifying EEG signal. Amplifier module 132 receives input signals IN1, SREF, IN2, and SACT from respective electrodes 122, 124, 126, and 128 and generates two amplified signals CH1 and CH2. Amplified signal CH1 is an amplified version of the voltage difference between signals IN1 and SACT, and amplified signal CH2 is an amplified version of a voltage difference between signals IN2 and SACT. Signal SREF is a shared reference that the balance differential amplifiers require for accurate amplification of signals having amplitude in the microvolt range.

Offset circuit 134 converts the AC amplified signals CH1 and CH2 into strictly positive signals in a voltage range (e.g., 0 to 5V) required for control module 135.

Control module 135 converts the strictly positive signal into streams of digital samples of respective signals CH1 and CH2, packages the samples for transmission, and provides the samples to interface circuit 138. In an exemplary embodiment of the invention, control module 135 includes a microprocessor such as the Atmel AT90S8535 8-bit microcontroller, which has analog-to-digital conversion capabilities. The microprocessor executes firmware that can include packaging the data in a serial stream containing error detection and frame synchronizing codes.

In an exemplary embodiment, interface circuit 138 is a wireless transmitter capable of transmitting the brainwave data to feedback system 150 without being electrically connected to feedback system 150. The wireless interface use infrared, radio, or other transmission techniques. In the exemplary embodiment, interface circuit 138 implements a serial port protocol such as the protocol required for an RS-232 serial port implemented by the Linx HP series II transmitter module. Alternatively, a wire or bus can connect interface circuit 138 to feedback system 150 if a wireless interface is not required. In such cases, interface circuit 138 would generally include an isolation circuit to reduce the chance of the user receiving an electrical shock from a malfunction of feedback system 150.

Power module 136 includes a battery (e.g., a 9V battery) and power management electronics that provide and distribute power at the required voltages to amplifier module 132, offset circuit 134, control module 135, and interface circuit 138. Power module 136 and more generally data acquisition unit 110 are preferably low power systems that do not create an electrical shock hazard.

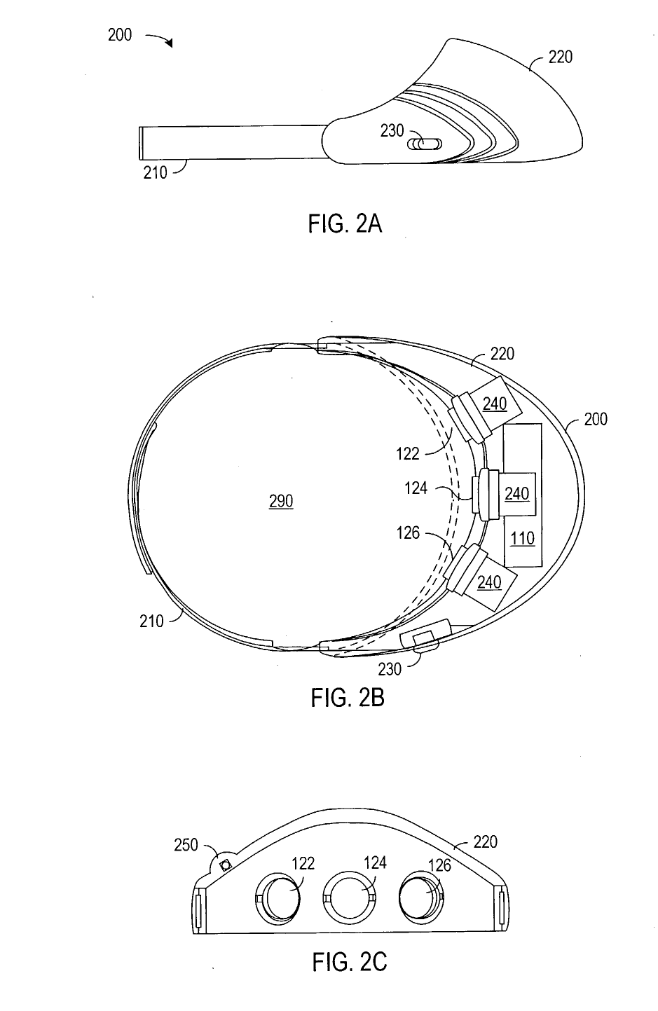

Data acquisition unit 110 can be contained in or mounted on a headset that is worn when using the system 100. FIGS. 2A, 2B, and 2C illustrate a headset 200 in accordance with an embodiment of the invention. FIG. 2A is a side view of headset 200, and FIG. 2B is a transparent top view of headset 200 when worn by a user 290. As shown in FIGS. 2A and 2B, headset 200 includes a headband 210 and a visor 220. FIG. 2C shows a view of an inner surface of visor 220 on which pliable electrodes 122, 124, and 126 are mounted.

Visor 220, which can be made of molded resin or other suitable material, contains space to accommodate the data acquisition unit 110, an on/off switch and indicator light 230, sockets 240 for pliable electrodes 122, 124, and 126, and a jack 250 for connecting wires or cables leading to ear electrode 128 (or feedback system 150 when a wireless interface is not used). In accordance with an aspect of the invention, sockets 240 in headset 200 properly position pliable electrodes 122, 124, and 126 on the forehead of the user 290 without requiring separate (and possibly inconsistent) placement of each electrode. Additionally, sockets 240 allow for easy removal of electrodes 122, 124, and 126 for cleaning or replacement.

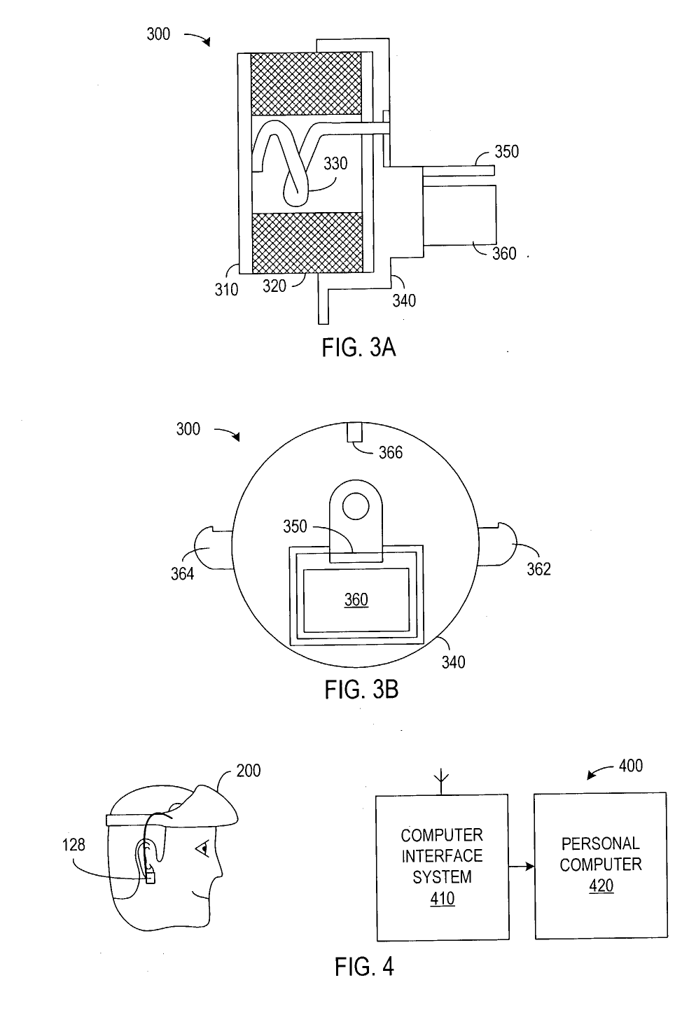

FIGS. 3A and 3B show an embodiment of a pliable electrode 300 in accordance with an embodiment of the invention. FIG. 3A is a cross-sectional view of pliable electrode 300, which includes a pliable conductive material 310, a compressible backing 320, a lead wire 330, and a molded structure 340. Conductive material 310 can be a conductive fabric (e.g., cloth impregnated with silver or silver chloride particles) or a conductive elastomer that is attached to compressible backing 320 and conductive lead 330.

Compressible backing 320 allows conductive material 310 to conform to the shape of the user's head and may be made of foam rubber or other spongy material capable of holding water when the electrode is dampened with plain tap water to improve conductivity. In the embodiment employing a conductive elastomer, compressible back 320 may be omitted if the conductive elastomer is sufficiently thick and compressible.

Lead wire 330 electrically connects conductive material 310 to an electrical contact 350 on the back of molded structure 340. An epoxy or adhesive (conductive or otherwise) can attach one end of lead wire 330 to conductive material 310 after the other end of lead wire 330 is soldered or otherwise electrically connected to contact 350.

Molded structure 340 forms a cup that contains compressible backing 320. The back of molded structure 340 as shown in FIG. 3B has a shape that matches a socket 240 and securely holds conductive material 350 in place. More specifically, a rectangular feature 360 on the back of molded structure 340 together with the position of electrical contact 350 and other features 362, 364, and 366 on molded structure 340 fix the orientation of electrode 300 when plugged into a matching socket 240.

Headset 200 can be used in a consumer EEG system in which data acquisition unit 110 communicates with either a standalone feedback system or a computer interface. FIG. 4 illustrates a consumer EEG system in which a headset 200 including a data acquisition unit that communicates with an interface 410 that relays data to a computer 420. In this configuration, interface 410 contains a receiver (e.g., a wireless receiver) to receive data from headset 200 and a standard interface (e.g., a RS-232, USB, or PCI interface) for communication with computer 420. Computer 420 executes software required to receive and process data from interface 410, to display feedback such as brainwave patterns, or to respond to the brainwave patterns to perform any user controllable function.

FIG. 1 illustrates components of a standalone feedback system 150 in the exemplary embodiment of the invention. As shown in FIG. 1, feedback system 150 includes a computer 160 that operates an audio output system 170, a screen I/O system 180, and a data I/O system 190. In presentation player 150 of FIG. 1, computer 160 has an interface circuit 163 that is compatible with interface circuit 138 and receives the data for the two channels of EEG signals. In an exemplary embodiment of the invention, interface circuit 163 is a wireless interface, and computer 160 is a system such as a model PCM-5820 available from Advantech or a custom processor board with processing power comparable to a personal computer.

Computer 160 contains several conventional interfaces including an audio port 164, a serial port 166, a video port 167, and a parallel port 168. In the embodiment of FIG. 1, computer 160 uses audio port 164 to drive an amplifier 172 in audio system 170, and amplifier 172 drives a speaker 174. Optionally, audio system 170 can further include a wave player that receives WAV data for a presentation.

Serial port 166 and video I/O interface 167 control screen system 180. In the illustrated embodiment, screen system 180 includes a touch screen 182 and a display screen 184. Display screen 184 can be an LCD screen or other device that provides visual information including but not limited to a representation of brain activity, control information, and video portions of presentations. The user can operate touch screen 182 to control operation of feedback system 150. Touch screen 182 and display screen 184 are examples of compact systems for input of control data and output visual information, but embodiments of the invention are not limited to I/O devices or displays of these types.

Parallel port 168 implements an interface for an external device 190. In an exemplary embodiment of the invention, external device 190 is a CD player with time scaling capabilities such as the “CD M200R Super Learning Compact Disk Player” available from SSI Corporation of Japan. Data I/O system 190 may alternatively include any peripheral or data storage device that can feedback unit 150 can control.

Computer 160 executes software or firmware routines from a memory such as a flash card 162. The firmware implements an operating system and the functions of feedback unit 150. The functions of feedback system 150 can vary widely depending on the application.

In one embodiment of the invention, feedback system 150 serves primarily or exclusively to provide biofeedback to the user. Computer 160 executes firmware to create a multimedia presentation from the EEG data. Since computer 160 is not limited to being a low power system, the multimedia presentation can include real time or frequently updated color video and sound representing the EEG signal. An exemplary embodiment of the feedback unit provides a high-resolution active matrix display with 16-million colors and a touch screen user interface in a self-contained device with no moving parts.

In another embodiment of the invention, feedback system 150 provides the biofeedback to the user and further implements a presentation system with time scaling capabilities. For a presentation system, computer 160 further includes a data I/O system (not shown) such as a CD drive, and computer 160 accesses from the data I/O system presentation data that may be unrelated to brain wave activity. Computer 160 can then time scale and play the presentation through audio system 170 and screen system 180. Other routines can simultaneously provide the video and audio according to the EEG data to permit a user to observe the current brainwaves while listening to a time-scaled presentation.

Computer 160 can further analyze the EEG data and change operating parameters such as the volume or the time scale of external device 190 or to select among available presentations. EEG signal control can further be applied to any function that computer 160 implements. In particular, the user can control the playback of 3D animation and audio (speed, play/pause, sequencing, etc.); play a software game involving object maneuvering, role playing, or mental strategy; or operate an electronic or mechanical system under the control of computer 160.

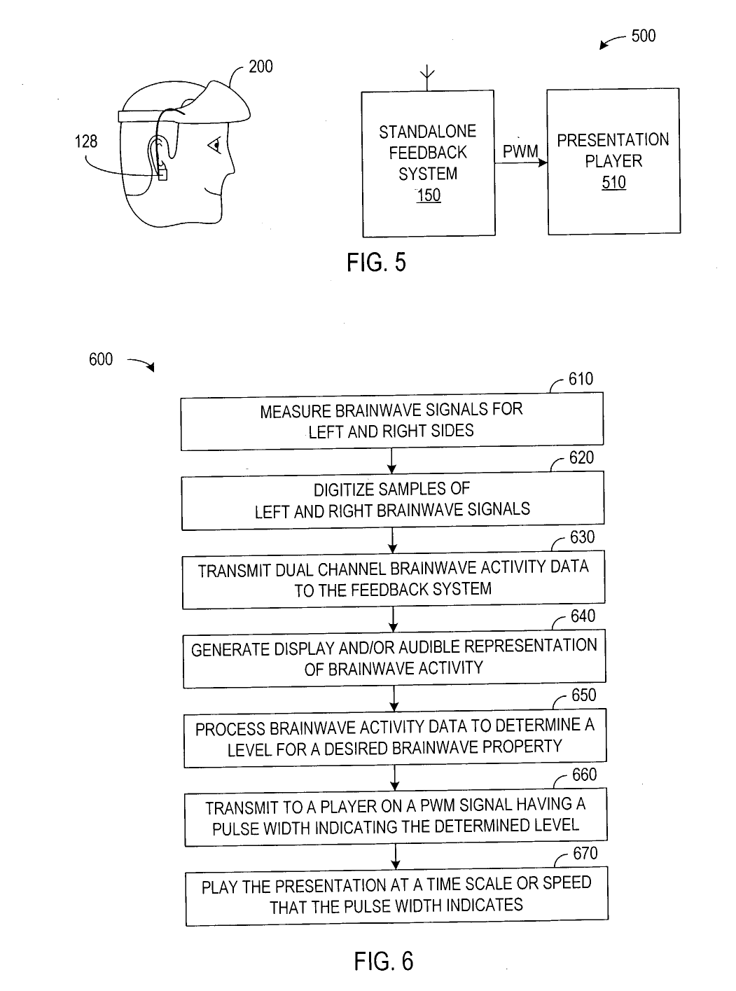

FIG. 5 illustrates a consumer EEG system having a headset 200 including a data acquisition unit that communicates with a standalone feedback system 150. In this configuration, feedback system 150 receives and interprets data representing brainwave activity and provides the user with a visual and/or audio representation of brainwave activity. Headset 200 and feedback system 150 can be used as a complete system if the user is only interested in observing brainwave activity.

Feedback system 150 of FIG. 5 further has the capability to control external devices such as a presentation player 510. When feedback system 150 is connected to an external device, feedback system 150 determines the characteristics of the brain activity data from headset 200 and generates a control signal that changes the operating parameters of the external device according to the determined characteristics. For presentation system 510, the control signal can turn presentation system 510 on or off or set operating parameters such as the playback speed, the volume, or the track being played.

FIG. 6 illustrates a process 600 for operation of a consumer EEG system such as illustrated in FIG. 1 to use brainwave activity for control of the speed or time scale of a presentation player. Data acquisition unit 110 in step 610 measures the left and right brainwave signals that are sampled and digitized in step 620 and transmitted to feedback system 150 in step 630.

Feedback system 150 processes the brainwave activity data and in step 640 generates a display representing the brainwave activity. For example, digital frequency filtering of the left and right digital signals can generate alpha, beta, and theta wave patterns for the left and right of the user's brain. Feedback system 150 can display all, some, or none of these patterns on display screen 184.

Further processing of the left and right brain activity data in step 650 determines a level of brainwave activity meeting a desired criterion. The level can depend on any desired characteristic of the brainwave activity data. In particular, the mean frequency of the brainwaves, the amplitude of the brainwave signal component in a selected frequency band such as the amplitude for alpha, beta, or theta waves, or synchronicity between the left and right brainwave activity signals.

One example of a possible criterion for level determination in step 650 is the average amplitude of alpha wave activity. The α-wave amplitude can have a range of values. Any α-wave amplitude above a maximum threshold voltage (e.g., 20 μV) can cause the level to be set to a maximum value. Smaller a-wave amplitudes down to a minimum threshold cause the level to be assigned lower values. Amplitudes below the minimum threshold voltage (e.g., 1 μV) or that fail to meet requirements such as a minimum duration above the minimum threshold or synchronicity between left and right brainwave signals result in the level being assigned the minimum level.

Another criterion for level determination in step 650 is synchrony between brain activity on the left and right sides of the user's brain. Software executed in feedback system 150 can quantify synchrony or similarity between EEG signals for the left and right hemispheres of the user's brain and use this information to set the level, e.g., for control the playback speed of a presentation system. Many researchers in the learning theory and peak performance training fields feel that synchronizing left and right brain states enhance one's ability to perform and learn. Therefore, the ability to assimilate verbally presented material at increased playback rates may be facilitated by left right synchrony training and feedback. Three example methods of assessing brain synchrony are disclosed below.

A first test analyzes a specified broad frequency band to determine the frequency with the highest amplitude in the left brain activity signal and the frequency with the highest amplitude in the right brain activity signal. The two frequencies are then evaluated to identify narrower frequency ranges such as theta (3-7 Hz), alpha (8-12 Hz), or beta (16-22 Hz) that characterize the two frequency signals. For example, within a broad frequency range of 2 to 30 Hz if the frequency with the greatest amplitude for the left hemisphere is say 8 Hz and the greatest amplitude for the frequency in the right hemisphere is 12 Hz, brain wave activity would be scored as synchronous in the alpha range.

A second test analyzes a specified broad frequency band to identify the frequency with the highest amplitude in left brain activity signal and the frequency with the highest amplitude in right brain activity signal. The two frequencies are then compared. For example, within a frequency band of 2 to 30 Hz if the frequency with the greatest amplitude, say 10 Hz, is the same at the left and right brain signals, brain activity is scored as synchronous, and the level is set to the highest value. This differs from the first analysis method which judges the brain activity as synchronous when the two frequencies merely fall within the same brainwave frequency bands such as theta, alpha, and beta.

A third test analyzes a specified frequency band and determines the peak frequency of the left and right brain wave signals and then determines whether the signs of the phase angles at the peak frequency are equal (i.e., either both positive or both negative). Brain activity is scored as synchronous if the frequency with the highest amplitude in both left and right hemispheres are the same and the phase angles have the same sign.

Software executed in feedback system 150 can implement many other measures or tests of brain synchrony. For example, feedback system can measure the correlation and coherence of the left and right brainwave signals to quantify brain synchrony. Feedback system then uses these synchrony measurement results to control the level, which controls an operating parameter such as the playback speed of a CD player.

The level determination 650 can use any or all of the described synchrony tests or other synchrony analysis in setting a level. For example, the level can have four values. The level has: a lowest value if the peak frequencies of the left and right brain activity signals are not of the same brainwave type theta, alpha, or beta; a second value if the peak left and right frequencies differ but are of the same brainwave type; a third value if the peak left and right frequencies are the same but differ in the sign for the FFT phase angles; and a highest value if the peak left and right frequencies are the same and have the same sign of the FFT phase angles.

The level can be set instead according to the user maintaining brain waves that pass a specific test for a required period of time. For example, the level can be set to a highest value only if the user maintains synchronous brain activity test over a required period of time. For example, brain activity falling out of synchrony may indicate that a presentation is being played at too fast of a speed, and step 650 could then reduce the level to reduce the playback speed.

Feedback system 150 uses the determined level to control an operating parameter of a presentation being played. In process 600, step 660 generates a pulse width modulated (PWM) signal for control of data I/O system 190. The PWM signal has duty cycle or pulse width that represents the determined level from stem 650. In an exemplary embodiment of the invention, external device 190 contains a CD, DVD, or other media on which an audio presentation is stored, and the PWM signal controls the playback speed or time scale of the audio presentation. (The recorded media may further include video or still images that are synchronized and played back with the audio presentation.)

One way for the presentation player or any other device to interpret the PWM signal is through integration of the PWM signal to generate a DC signal having a voltage that depends on the duty cycle of the PWM signal. Integration of a PWM is generally best if the PWM signal has a carrier frequency of about 10 kHz or more. The presentation player can either use the integrated voltage as an analog control signal or perform an analog-to-digital conversion to determine the playback speed.

Another way for interpreting the PWM signal is to digitally sample the PWM signal and determine the duty cycle from the samples. With this technique, a low frequency PWM signal (e.g., a 1-Hz signal) can be sampled 100 times per cycle (e.g., at 100 Hz), and the number of samples having a high level indicates the duty cycle. The PWM control signal has the advantages that a single line is sufficient for control and a complex protocol or signal synchronization is not required. The feedback system and the external device can thus operate asynchronously without complicated signal protocols.

With either PWM technique, the playback speed or time scale of the presentation corresponds to the duty cycle of the PWM signal which in turn depends on a level calculated from the brainwave data. For one of the exemplary criterion, the presentation playback speed is at a rate that maintains a desired level of synchrony between brain activity on the left and right side of the user's brain. If brain activity synchrony changes, the presentation player can automatically change to time scale that keeps brain activity synchronous, which may provide optimal learning efficiency. The presentation player can use other determined characteristics of brain activity to adapt to the user's needs.

Another criterion noted above sets the level according to the amplitude of brain activity in a particular frequency band. If a user can develop voluntary control of the amplitude of the user's alpha waves, for example, the user can control the presentation speed (or an operating parameter of another external device) using only brain activity. Other characteristics of the brainwave data can also be used for direct control of other parameters of the presentation.

Although the invention has been described with reference to particular embodiments, the description is only an example of the invention's application and should not be taken as a limitation. For example, although much of the above description is directed to control of presentation systems based on brainwave activity, similar control techniques can be applied to other devices. Various other adaptations and combinations of features of the embodiments disclosed are within the scope of the invention as defined by the following claims.

What is claimed is:

1. A system comprising:
headgear;
a first sensing electrode mounted on the headgear so as to contact a forehead of a user wearing the headgear, the first sensing electrode being a pasteless electrode that is pliable;
a second sensing electrode for use as an electrical contact to the user; and
an amplifier connected to the first and second sensing electrodes, the amplifier producing a signal that depends on a difference between potentials of the first and second sensing electrode.

2. The system of claim 1, further comprising a wireless transmitter connected to the amplifier to transmit data representing the signal.

3. The system of claim 2, further comprising a feedback unit that includes:
a wireless receiver capable of receiving the data from the wireless transmitter; and
a processor coupled to receive the data from the wireless receiver.

4. The system of claim 1, wherein the second sensing electrode comprises a clip suitable for clipping to an ear of the user.

5. The system of claim 4, further comprising a third sensing electrode, the third electrode being a pasteless, pliable electrode that is mounted on the headgear so as to contact the forehead of the user wearing the headgear.

6. The system of claim 5, wherein the amplifier is connected to the first, second and third electrodes.

7. The system of claim 6, further comprising a second amplifier producing a signal that depends on a difference between potentials of the third and second sensing electrodes.

8. The system of claim 5, further comprising a fourth electrode, the fourth electrode being a pasteless, pliable electrode that is mounted on the headgear so as to contact the forehead of the user wearing the headgear, wherein the fourth electrode contacts a portion of the forehead that is between portions that the first and third electrodes contact.

9. The system of claim 8, wherein the amplifier is a dual channel differential balanced amplifier connected to use a signal from the fourth electrode as a shared reference.

10. The system of claim 1, wherein the first electrode is a dry electrode.

11. The system of claim 1, wherein the first electrode is dampened with water.

12. The system of claim 1, wherein the signal comprises a brain activity signal.

13. The system of claim 1, wherein the headgear comprises a socket into which the first electrode is plugged for use, the first electrode being removable from the socket.

14. The system of claim 13, wherein the first electrode comprises:
a rigid structure;
a compressible backing in the rigid structure; and
a conductive material attached to the compressible backing.

15. The system of claim 14, wherein the conductive material comprises a conductive fabric.

16. The electrode of claim 14, wherein the conductive material comprises a conductive rubber material.

17. The electrode of claim 14, wherein the rigid structure includes a cup in which the compressible backing resides, the cup having a shape that fits into the socket.

18. An electrode for an EEG, comprising:
a rigid structure;
a compressible backing in the rigid structure; and
a conductive material attached to the compressible backing.

19. The electrode of claim 18, wherein the conductive material comprises a conductive fabric.

20. The electrode of claim 18, wherein the conductive material comprises a conductive rubber material.

21. The electrode of claim 18, wherein the compressible backing comprises foam rubber material.

22. The electrode of claim 18, wherein the rigid structure includes a cup in which the compressible backing resides.

23. An EEG system comprising a sensing electrode made of a conductive rubber material.

24. The EEG system of claim 23, further comprising a headset in which the sensing electrode is mounted so as to contact a user's head.

25. A presentation system comprising:
a player that is capable of playing presentations at an adjustable time scale; and
a sensor connected to sense brain activity of a user and to provide to the player a control signal that depends on the brain activity sensed.

26. The system of claim 25, further comprising a headset on which the sensor is mounted, the headset positioning the sensor in proximity to the head of the user.

27. The system of claim 25, wherein the control signal comprises a pulse width modulated signal.

28. The system of claim 27, wherein a pulse width of the pulse width modulated signal controls a time scale at which the player plays a presentation.

29. The presentation system of claim 25, wherein the sensor comprises:
a headset containing a data acquisition unit; and
a feedback system that receives and processes a brain activity signal from the data acquisition, the feedback system generating an observable representation of the brain activity of the user.

30. The system of claim 25, wherein the control signal has a level that depends on synchrony between brain activity measured for a left side of a user's head and brain activity measured for a right side of the user's head.

31. A method for controlling a presentation system comprising:
measuring a left signal representing brain activity from a left side of a user's head while the user senses a presentation;
measuring a right signal representing brain activity from a right side of the user's head while the user senses the presentation; and
setting a play rate of the presentation according to synchrony between the first and second signals.

32. The method of claim 31, further comprising measuring synchrony between the left and right signals.

33. The method of claim 32, wherein measuring synchrony comprises:
identifying a left frequency that corresponds to a frequency component that has the greatest amplitude within a selected band of the left signal;
identifying a right frequency that corresponds to a frequency component that has the greatest amplitude within the selected band of the right signal; and
comparing the left and right frequencies.

34. The method of claim 33, wherein comparing comprises determining if the left frequency has a brainwave type that matches a brainwave type of the right frequency.

35. The method of claim 33, wherein comparing comprises determining whether the left frequency is equal to the right frequency.

36. The method of claim 35, wherein comparing further comprises determining whether the component corresponding to the left frequency has a phase angle with a sign that is equal to a sign of a phase angle of the component corresponding to the right frequency.
